(19) United States
(12) Patent Application Publication
(10) Pub. No.: US 2021/0161375 A1
(43) Pub. Date: Jun. 3, 2021

(54) Digital Colposcope for Self-Examination

(71) Applicant: Naser Omer Ali, Minneapolis, MN (US)

(72) Inventor: Naser Omer Ali, Minneapolis, MN (US)

(21) Appl. No.: 16/820,783

(22) Filed: Mar. 17, 2020

Related U.S. Application Data
(60) Provisional application No. 62/974,520, filed on Dec. 3, 2019.

(51) Int. Cl. A61B 1/303; A61B 1/05; A61B 1/00

(52) U.S. Cl. CPC A61B 1/303; A61B 1/05; A61B 1/00009; A61B 1/00016; A61B 1/00045; A61B 1/0008

(57) ABSTRACT

A lightweight and portable digital colposcope that could allow a subject to self-examine her vagina or cervix. The digital colposcope comprises an elongate main body for insertion into the subject's vagina. There is a digital camera module contained in the main body. The main body could also hold a wireless transmitter to send images to an external display device. The digital colposcope could have a system of self-expanding scaffold, stretchable protective membrane, and slidable sheath to widen the vaginal canal for better visualization.

### TECHNICAL FIELD

This invention relates to colposcope devices for examining a subject's vagina and cervix.

### BACKGROUND

A colposcope is a medical device used to examine a patient's vagina and cervix for detecting abnormalities such as cancerous growths or infection. Conventional colposcopes are expensive equipment that are bulky and heavy. Thus, they are not easily portable. Moreover, colposcopes are used in conjunction with a speculum to expand the vaginal canal to enable better viewing of the cervix. The speculum requires an external user for proper insertion.

Thus, conventional colposcope exams are performed in a clinical facility (e.g. doctor's office). There is a need for colposcopes that are lightweight, low cost, easily portable, and easy-to-use to allow for self-examination outside of a clinical facility (e.g. at home). With the appropriate technology, it is possible that a user could self-perform an examination on herself.

### SUMMARY

My invention addresses this need for a device that allows self-examination of female internal genitalia, e.g. cervix and vagina. In one embodiment, my invention is a digital colposcope that could be used for self-examination. The digital colposcope comprises an elongate main body having a distal end and a proximal end. The main body could have any suitable dimension for probing into a subject's vaginal canal. In some embodiments, the main body has a length in the range of 10 to 40 cm; and in some cases, in the range of 15 to 35 cm. In some embodiments, the main body has a width in the range of 0.5 to 3.0 cm; and in some cases, in the range of 1.0 to 2.5 cm.

There is a digital camera module contained in the main body and has a lens cover at the distal end of the main body. The main body could also hold a wireless transmitter to send images to an external display device (e.g. a smartphone).

There is a self-expanding scaffold around the main body. The scaffold is designed to have a smaller compressed configuration, but self-expand in a radially outward direction when the compressive force is released. There is a stretchable protective membrane over the self-expanding scaffold. This protective membrane serves to avoid roughness or abrasion of the scaffold against the vaginal canal.

A slidable sheath over the protective membrane serves to radially compress the self-expanding scaffold when fully advanced to its distal position. Retraction of the slidable sheath in a proximal direction allows the scaffold to self-expand in a radially outward direction, thereby inflating the protective membrane. The slidable sheath could comprises an annular ridge at its distal end to facilitate sliding of the sheath over the protective membrane.

The self-expanding scaffold could have any suitable configuration or framework that acts like a spring to recover its expanded configuration when the compressing force is removed. For example, the self-expanding scaffold could be a coil or mesh. In some embodiments, the scaffold comprises a plastic material, stainless steel, or a shape memory metal such as nitinol. The digital colposcope could comprise a light source to illuminate the vaginal canal. For example, it could have LED illumination at the distal end of the main body.

In another embodiment, my invention is a method of performing vaginal or cervical examination using the above-described digital colposcope of my invention. The method could be performed by the subject herself (self-examination) or by another person (e.g. an assistant). The method comprises inserting (or self-inserting) the digital colposcope into the subject's vagina with the distal end of the main body pointing into the vagina. The main body of the digital colposcope is advanced into the vagina towards the cervix. Images of the vagina and cervix are acquired for analysis. The images may be transmitted to an external display device (e.g. a smartphone) via wired or wireless communication.

In some embodiments, the method further comprises, as the distal end of the main body approaches the cervix, retracting the slidable sheath in a proximal direction. This allows the scaffold to self-expand in a radially outward direction, thereby inflating the protective membrane to widen the vaginal canal passageway. For removal of the colposcope, the sheath could be advanced distally to downsize the expanded membrane. This could help in removal of the colposcope out of the vaginal canal.

In another embodiment, my invention is yet another type of digital colposcope with a display screen. This digital colposcope comprises an elongate main body having a distal end and a proximal end. The main body could have the dimensions given above. The main body could have a digital camera, lens cover, wireless transmitter, or other components described above. The digital colposcope could comprise a light source to illuminate the vaginal canal. For example, it could have LED illumination at the distal end of the main body.

The digital colposcope further comprises a display panel comprising a display screen. The colposcope is configured such that the display screen faces more towards the distal direction than towards the proximal direction relative to the main body of the colposcope. The display panel could be attached to the main body in any suitable way. In some embodiments, at the proximal end of the main body, there is a connecting arm that connects the display panel to the main body. Such connecting arm could be fixed or flexible.

In fixed configurations, the angle between the central longitudinal axis of the main body and the plane of the display panel could be in the range of 0 to 90°; and in some cases, in the range of 30 to 75°. In flexible configurations, the range-of-motion for the display panel relative to the central longitudinal axis of the main body could be encompassed within the range of 0 to 90° (e.g. the possible range-of-motion could be 30 to 60°); and in some cases, encompassed within the range of 30 to 75°.

In another embodiment, my invention is a method of performing vaginal or cervical examination using the above-described digital colposcope with display screen of my invention. The method could be performed by the subject herself (self-examination) or by another person (e.g. an assistant). If used for self-examination, the colposcope could be configured such that the display screen faces towards the subject's eyes.

The method comprises inserting (or self-inserting) the digital colposcope into the subject's vagina with the distal end of the main body pointing into the vagina. The main body of the digital colposcope is advanced into the vagina towards the cervix. Images of the vagina and cervix are acquired for analysis. The images may be transmitted to an external display device (e.g. a smartphone) via wired or wireless communication.

In fixed configurations, the angle of insertion may be adjusted for better viewing of the display screen. In flexible configurations, the connecting arm may be adjusted to angle the display panel for better viewing of the display screen.

In some embodiments, the above-described digital colposcopes could have other sensor elements such as a urine chemistry sensor. The patient could urinate on the sensor to diagnose abnormalities that manifest in urinalysis. The digital colposcopes of my invention could provide better service and quality for home or clinical setting (e.g. hospital or doctor office). Using this digital colposcope could be used to detect abnormalities such as cancerous growths or infection. This digital colposcope could also be used to examine the cervix to assess how a pregnancy and labor is progressing. The digital colposcope could transmit the images to a screen device (e.g. smartphone) and could also assess the images for diagnosis.

### BRIEF DESCRIPTION OF THE DRAWINGS

FIG. 1 shows a side view of an example digital colposcope of the invention.

FIGS. 2A and 2B show different perspective views of the distal portion of the digital colposcope shown in FIG. 1.

FIGS. 3A-3D show a “build-up” view of the digital colposcope shown in FIG. 1.

FIG. 4 shows a side view of the digital colposcope with a cutaway window.

FIGS. 5A-5C show the operation of the digital colposcope.

FIGS. 6A and 6B show an example of how the digital colposcope could be used.

FIG. 7 shows a perspective view of another example digital colposcope of my invention.

FIGS. 8A and 8B show an example of how the digital colposcope of FIG. 7 could be used.

### DETAILED DESCRIPTION

To assist in understanding the invention, reference is made to the accompanying drawings to show by way of illustration specific embodiments in which the invention may be practiced.

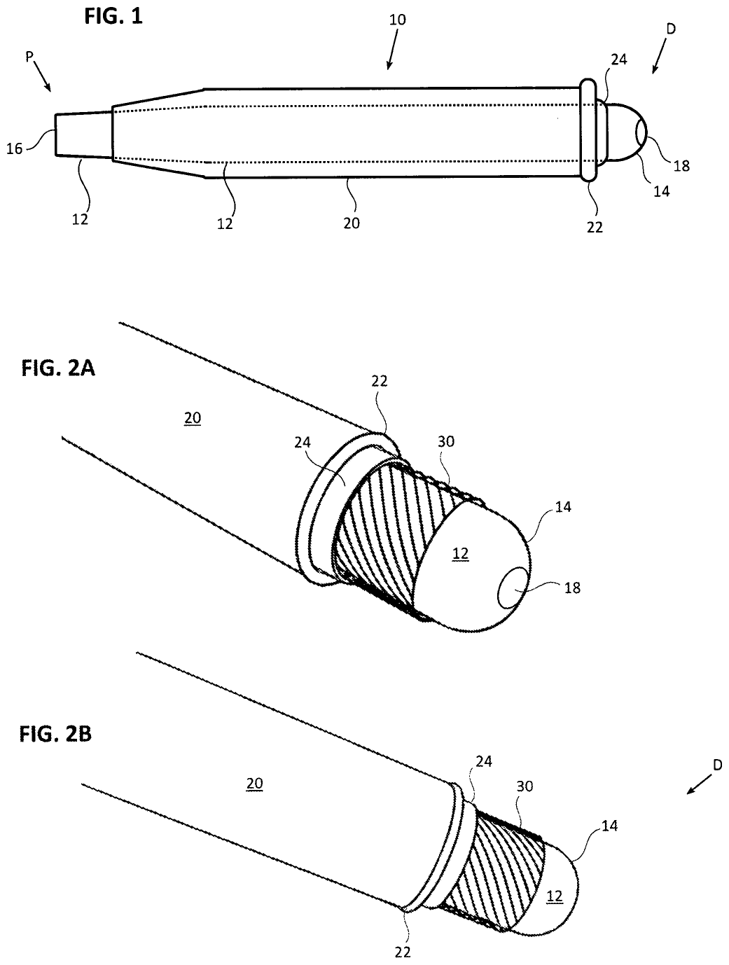

FIG. 1 shows a side view of an example digital colposcope 10 of the invention. The digital colposcope 10 comprises an elongate main body 12. To specify orientation, the distal direction is indicated by the reference label “D” and the proximal direction is indicated by the reference label “P.” At the distal end 14, there is a lens cover 18 for the digital camera. Also behind the lens cover 18 are LED lights for illumination. A partial view of the distal end of the stretchable balloon 24 is visible underneath the slidable sheath 20 covering over the balloon 24. The slidable sheath 20 is fully advanced to hold the spiral coil 30 in place (not seen in this view). The distal end of the sheath 20 has an annular ridge 22 to facilitate sliding of the sheath 20 over the balloon 24. The proximal end 16 is also shown. The digital colposcope 10 uses a wireless transmitter to send images to any suitable display device, e.g. a smartphone or display monitor.

FIGS. 2A and 2B show different perspective views of the distal portion of the digital colposcope 10. Over the main body 12, there is a spiral coil 30, which is in a compressed configuration, but ready to self-expand outwardly in a radial direction. The protective balloon 24 is shown in truncated, cutaway view to show how it covers over spiral coil 30. The sheath 20 covers over the protective balloon 24 and the spiral coil 30 to restrain the radial self-expansion of the spiral coil 30. Here, the sheath 20 is retracted slightly, but for better visual clarity, the protective balloon 24 and the spiral coil 30 are shown in their unexpanded configuration. In actual use, the retraction of sheath 20 would allow the spiral coil 30 to expand, along with the protective balloon 24.

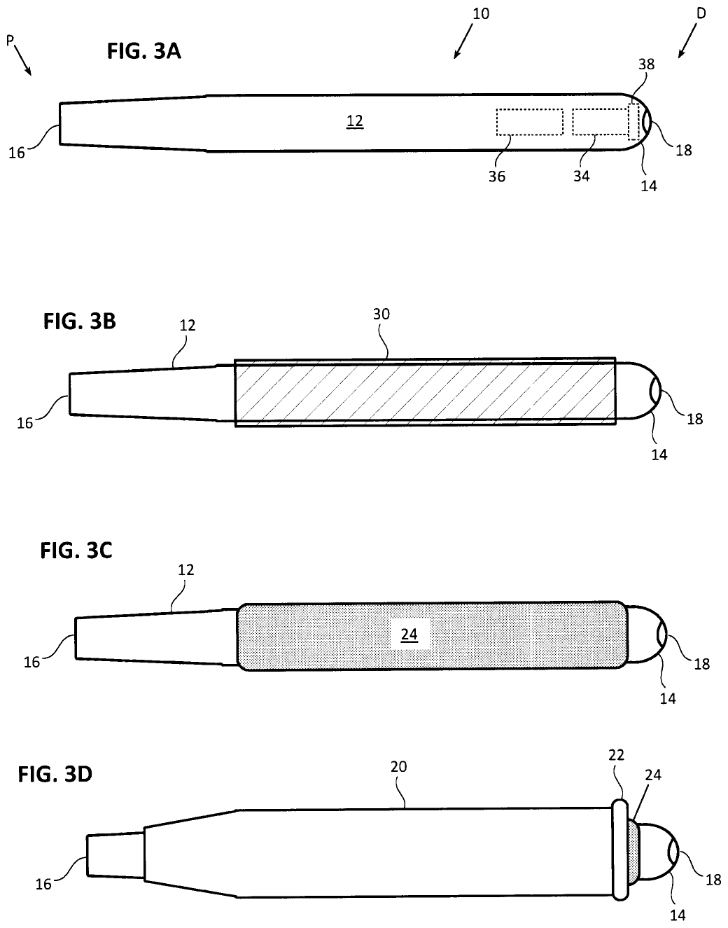

FIGS. 3A-3D show a “build-up” view of the digital colposcope 10, i.e. showing the device in a series of layers. FIG. 3A shows the elongate main body 12 of the digital colposcope 10 with the distal end 14 and the proximal end 16. The main body 12 houses the various electronic components for operation of the digital colposcope 10. For example, it may house the digital camera module, battery power supply, LED illumination, wireless communication (e.g. Bluetooth transmitter), user controls (e.g. buttons, switches, etc.) and so on. This view shows the digital camera module 34, wireless transmitter 36, and LED lights 38. Some of the electronic and mechanical components that could be used in the main body 12 are described in patent publications US 2019/0150725 (Ramanujam et al.) and WO 2019/070998 (Duke University), which are incorporated by reference herein. At the distal end tip of the main body 12 is the lens cover 18 for the digital camera and LED illumination lights.

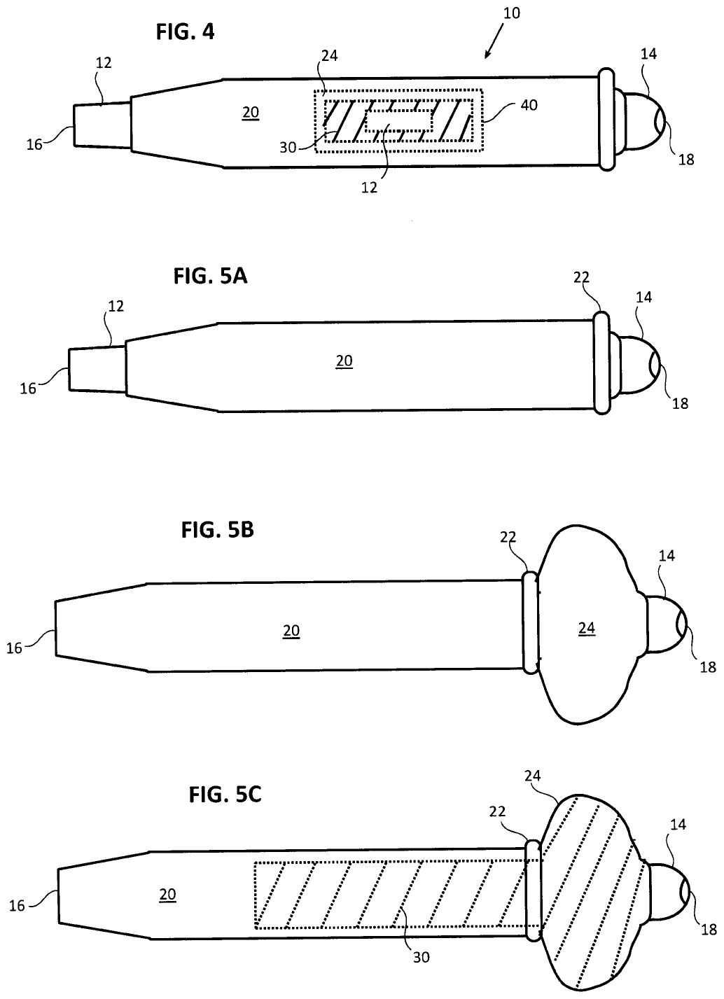

FIG. 3B shows the spiral coil 30 extending over the main body 12. The spiral coil 30 is in a radially compressed configuration that is capable of self-expanding to a larger diameter. The spiral coil 30 may be made of any suitable material, such as stainless steel or plastic. FIG. 3C shows the protective balloon 24 covering over the spiral coil 30. The function of the protective balloon 24 is to reduce abrasion against the vaginal mucosa. FIG. 3D shows the slidable sheath 20 covering over the protective balloon 24. The function of the sheath 20 is to restrain the radial self-expansion of the spiral coil 30. The slidable sheath 20 can be retracted proximally or advanced distally to control the expansion of the balloon 24 and coil 30. FIG. 4 shows a side view of the digital colposcope 10 with a cutaway window 40 to expose the underlying layer structures. In the cutaway window 40, the overlapping layers of the slidable sheath 20, the balloon 24, the spiral coil 30, and main body 12 are all visible.

FIGS. 5A-5C show the operation of the digital colposcope 10. In FIG. 5A, the digital colposcope 10 is provided in a compact configuration with the slidable sheath 20 fully advanced to its distal position. In FIG. 5B, the sheath 20 is retracted in the proximal direction. This exposes a portion of the protective balloon 24 and releases the spiral coil 30, allowing it to unwind and expand outward against the balloon 24. FIG. 5C shows a see-through view of the spiral coil 30 with its distal segment unwound to inflate the balloon 24.

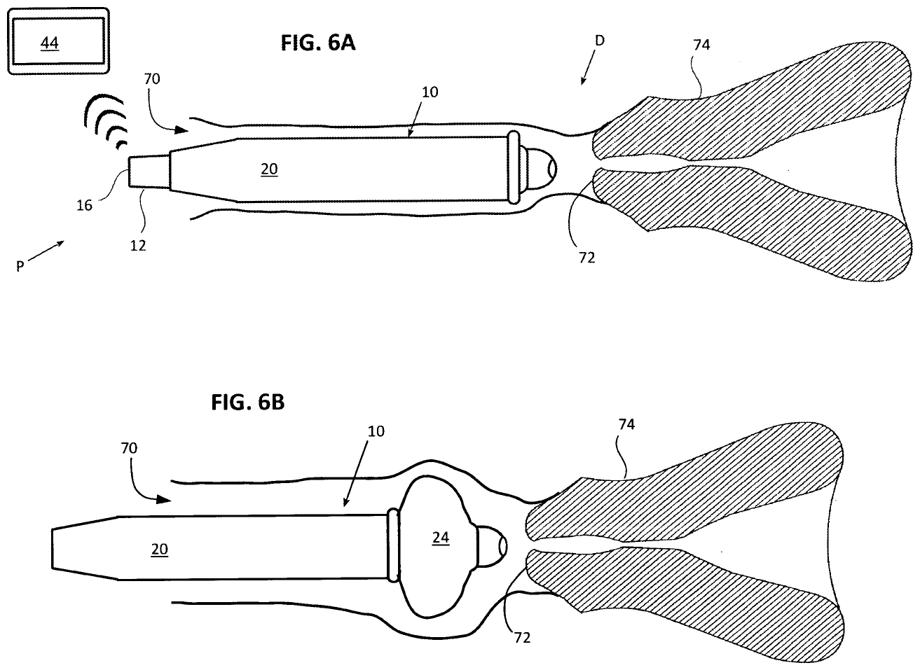

FIGS. 6A and 6B show an example of how the digital colposcope 10 could be used. The relevant anatomy here is the uterus 74, the cervix 72, and the vaginal canal 70. Initial insertion of a vaginal speculum is optional, but not necessary. As seen in FIG. 6A, the user self-inserts the colposcope 10 into her vaginal canal 70 with the distal end pointing in. The user views the camera images on a mobile smartphone 44, which receives the images via wireless transmission from the colposcope 10. Alternatively, the colposcope 10 may be connected to the smartphone via a cord for image data transmission, e.g. a USB port. The user continues to advance the colposcope 10 through the vaginal canal 70 towards cervix 72. Because the contours of the vaginal canal 70 may not allow adequate visualization of the passageway, the user retracts the sheath 20 in a proximal direction.

As shown in FIG. 6B, this exposes the protective balloon 24 and releases the spiral coil, allowing it to expand outward against the balloon 24. Inflation of balloon 24 in this manner widens the passageway to facilitate visualization of the cervix 72. Video or still images of the cervix 72 may be taken and recorded. Clinical assessment of the images could be performed automatically by artificial intelligence software, review by a clinician, or combination thereof (e.g. computer-assisted diagnosis). The artificial intelligence software may reside on the colposcope 10, on a remote device (e.g. a smartphone app), or a combination thereof.

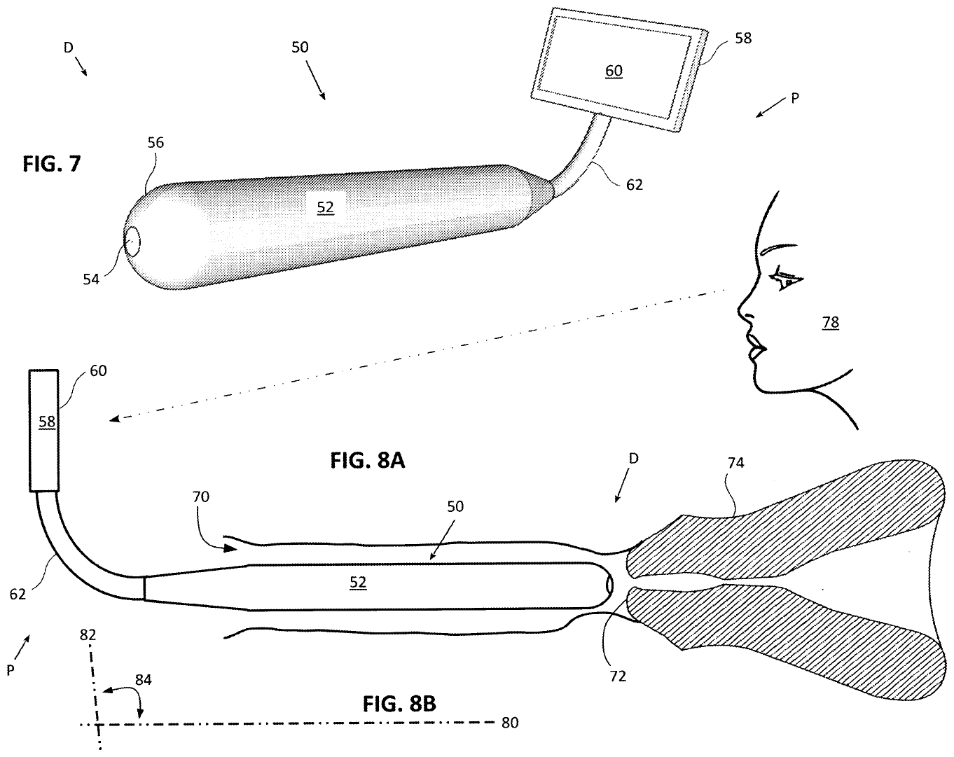

FIG. 7 shows a perspective view of another example digital colposcope 50 of the invention. The digital colposcope 50 comprises an elongate main body 52 with a distal end 56. To specify orientation, the distal direction is indicated by the reference label “D” and the proximal direction is indicated by the reference label “P.” At the distal end 56, there is a lens cover 54 for the digital camera and LED illumination. At the proximal end, there is a display panel 58 having a display screen 60 to display images acquired from the digital camera. The colposcope 50 is configured such that display screen 60 faces towards the distal direction, i.e. towards the self-examining user. The display panel 58 is connected to the main body 52 by a flexible connector arm 62, which allows the display panel 58 to be adjusted for better viewing by the user. The display screen 60 may have a touchscreen interface.

FIGS. 8A and 8B show an example of how the digital colposcope 50 could be used. Initial insertion of a speculum is optional, but not necessary. As shown in FIG. 8A, the user 78 self-inserts the colposcope 50 into her vaginal canal 70 with the distal end pointing in. The user 78 views images from the digital camera of the colposcope 50 on the display screen 60 of the panel 58. Because the display screen 60 is facing towards the user 78, this facilitates easier viewing by the user 78. The user 78 continues to advance the colposcope 50 through the vaginal canal 70 towards cervix 72. Video or still images of the cervix 72 may be taken and recorded. Clinical assessment of the images may be performed automatically by artificial intelligence, review by a clinician, or combination thereof (e.g. computer-assisted diagnosis). Clinical assessment of the images could be performed automatically by artificial intelligence software residing on the colposcope 50, on a remote device (e.g. a smartphone app), or a combination thereof.

FIG. 8B shows how to measure the angle between the display panel 58 and the main body 52. The main body 52 has a central longitudinal axis 80. This intersects with the crossing axis 82 of display panel 58 (on the imaginary plane upon which the display panel 58 resides). The angle 84 between axis 80 and axis 82 is considered the angle between the display panel 58 and the main body 52. This also applies in the alternate scenario where the connector arm 62 between display panel 58 and main body 52 is fixed.

In addition to the descriptions given above, the digital colposcope of the present invention could have any of various different sizes and shapes. The digital colposcope could be used for examining the internal or external genitalia of males or females. The digital colposcope could also detect or identify abnormalities and transmit the results (e.g. images or descriptive text) to a display screen in real-time. The digital colposcope could also have a sensor for urine analysis, which could be implemented by urinating on the sensor, wiping the sensor (e.g. with bathroom tissue), or inserting the sensor into the urinary tract. Again, the digital colposcope could also detect or identify abnormalities in the urine sample and transmit the results (e.g. images or descriptive text) to a display screen in real-time. In males, this could also be used in the relevant anatomy of male subjects, such as anus or penis.

The foregoing description and examples have been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A digital colposcope comprising:
an elongate main body having a distal end and a proximal end;
a digital camera module contained in the main body and having a lens cover at the distal end of the main body;
a self-expanding scaffold around the main body, wherein the scaffold self-expands in a radially outward direction;
a stretchable protective membrane over the self-expanding scaffold;
a slidable sheath over the stretchable membrane that serves to radially compress the self-expanding scaffold when fully advanced to its distal position.

2. The digital colposcope of claim 1, wherein the self-expanding scaffold is a coil or mesh.

3. The digital colposcope of claim 1, wherein the scaffold comprises stainless steel or nitinol.

4. The digital colposcope of claim 1, further comprising LED illumination at the distal end of the main body.

5. The digital colposcope of claim 1, wherein retraction of the slidable sheath in a proximal direction allows the scaffold to self-expand in a radially outward direction, thereby inflating the protective membrane.

6. The digital colposcope of claim 1, wherein the main body has a length in the in the range of 10 to 40 cm.

7. The digital colposcope of claim 1, wherein the main body has a width in the range of 0.5 to 3.0 cm.

8. The digital colposcope of claim 1, further comprising a wireless transmitter to send images to an external display device.

9. The digital colposcope of claim 1, wherein the slidable sheath comprises an annular ridge at its distal end.

10. A method of performing vaginal or cervical examination on a subject, comprising:
(a) having a digital colposcope that comprises:
an elongate main body having a distal end and a proximal end;
a digital camera module contained in the main body and having a lens cover at the distal end of the main body;
a self-expanding scaffold around the main body, wherein the scaffold self-expands in a radially outward direction;
a stretchable protective membrane over the self-expanding scaffold;
a slidable sheath over the stretchable membrane that serves to radially compress the self-expanding scaffold when fully advanced to its distal position.
(b) inserting the digital colposcope into the subject's vagina with the distal end of the main body pointing into the vagina;
(c) advancing the main body into the vagina;
(d) acquiring images of the vagina or cervix.

11. The method of claim 10, further comprising:
with the main body at least partially inside the vagina, retracting the slidable sheath in a proximal direction;
allowing the scaffold to self-expand in a radially outward direction, thereby inflating the protective membrane to widen the vaginal canal passageway.

12. The method of claim 10, further comprising transmitting the images to an external display device.

13. The method of claim 11, further comprising:
advancing the sheath distally to downsize the expanded membrane;
removing the colposcope out of the vaginal canal.

14. The method of claim 10, wherein the subject self-performs the examination.

15. The method of claim 10, wherein the examination is performed by someone other than the subject.

16. The method of claim 10, wherein the main body of the digital colposcope has a length in the in the range of 10 to 40 cm.

17. A digital colposcope comprising:
an elongate main body having a distal end and a proximal end;
a display panel comprising a display screen that faces more towards the distal direction than towards the proximal direction relative to the main body;
at the proximal end of the main body, a connecting arm that connects the display panel to the main body.

18. The digital colposcope of claim 17, wherein the connecting arm is flexible.

19. The digital colposcope of claim 18, wherein the range-of-motion for the display panel relative to the central longitudinal axis of the main body is encompassed within the range of 0 to 90°.

20. The digital colposcope of claim 17, wherein the connecting arm is fixed.
